(19) United States
(12) Patent Application Publication
(10) Pub. No.: US 2014/0142464 A1
(43) Pub. Date: May 22, 2014

(54) Systems and Methods for Surgery Rotating Needles and Therapy Treatments

(71) Applicants: Steven E. Harms, Fayetteville, AR (US); David H. Lewis, Fort Collins, CO (US); John P. Heinrich, Fort Collins, CO (US)

(72) Inventors: Steven E. Harms, Fayetteville, AR (US); David H. Lewis, Fort Collins, CO (US); John P. Heinrich, Fort Collins, CO (US)

(73) Assignee: HIGH PLAINS TECHNOLOGY GROUP, LLC, Fort Collins, CO (US)

(21) Appl. No.: 14/127,115

(22) PCT Filed: Jun. 18, 2012
(86) PCT No.: PCT/US12/42957

§ 371 Date: Dec. 17, 2013

Related U.S. Application Data
(60) Provisional application No. 61/498,402, filed on Jun. 17, 2011.

(51) Int. Cl. A61B 10/02; A61B 17/34

(52) U.S. Cl. CPC A61B 10/0283; A61B 17/3476
USPC 600/566; 606/180

(57) ABSTRACT

The inventive technology describes to a novel surgical device having a high-speed rotating hollow cutting needle capable of longitudinally cutting, sampling, removing and/or coring targeted tissue pathologies as well as the efficient collection and/or disposal of said tissue pathologies. In particular, the inventive technology may be suitable for application on lesions near the skin, and/or pathologies internal to the body, and perhaps even especially for procedures performed in conjunction with image guidance, particularly active X-ray, computerized tomography (CT), magnetic resonance imaging (MRI), and/or ultrasound scans, where the presence of a large electro-magnetic field may impose significant constraints on the design and operation of mechanical mechanisms. Additionally, the inventive technology encompasses a system of device assisted targeted delivery of a variety of therapeutic treatments to specific tissue pathologies, through, for example, adaptable therapeutic introducers coordinated with a rotating hollow cutting needle.

This application claims the benefit of and priority to U.S. Provisional Application No. 61/147,359 filed on Jun. 17, 2011. The entire specification and figures of the above-mentioned application is hereby incorporated, in its entirety by reference.

### TECHNICAL FIELD

Generally, this inventive technology relates to the field of surgical devices and device assisted therapeutic treatment delivery. More specifically, certain embodiments of the current inventive technology relate to a surgical device having a high-speed rotating hollow cutting needle capable of axially or longitudinally cutting, sampling, removing and/or coring targeted tissue pathologies as well as the efficient collection and/or disposal of said tissue pathologies. In particular, the inventive technology may be suitable for application on tissue, pathologies and/or lesions (the terms being interchangeable) near the skin, and/or internal to the body, and perhaps even especially for procedures performed in conjunction with real-time or approximately real-time image guidance, particularly active X-ray, computerized tomography (CT), magnetic resonance imaging (MRI), and ultrasound scans, where the presence of an electro-magnetic field may impose significant constraints on the design and operation of mechanical mechanisms. It should be noted that the aforementioned surgical device and associated techniques are equally applicable to animal as well as human subjects.

In addition certain embodiments of the inventive technology may also encompass novel methods and apparatus for the delivery of therapeutic treatments. More specifically, as will be detailed below, certain embodiments may allow for the targeted delivery of a variety of therapeutic treatments to specific tissue pathologies, perhaps through adaptable therapeutic introducers coordinated with said rotating hollow needle.

### BACKGROUND

A wide range of diagnostic techniques including X-ray CT, MRI, and ultrasound may be used to detect and diagnose lesions within various organs in the human body. If a lesion is detected, a sample of tissue may be taken for analysis using a conventional biopsy system. However, depending on the location and nature of the lesion and the exact medical circumstances, it may be advantageous to remove a larger portion of or even the entire lesion. As conventional surgery may not be practical under these conditions, a surgical device that can quickly, efficiently and even safely remove larger tissue volumes may be required. The ability to readily visualize the device under image guidance, the ability to minimize trauma, and perhaps even the ability to provide access for other types of therapy may be advantageous.

Existing devices known in the art are woefully deficient in achieving these broad objectives. For example, such traditional devices may tend to utilize an outer tube with a lateral cutting window in combination with an inner tube driven in either rotary or reciprocal fashion to cut tissue. Such devices are not truly surgical devices as they can only perhaps capture a small portion of a targeted lesion, and inefficiently at that, especially where the lesion is deep with in the body, or is encapsulated and/or transiently positioned. Examples of these traditional efforts are illustrated in U.S. Pat. Nos. 4,011,869, and 5,423,844, hereby incorporated by reference herein. The limitations of these approaches may be evident based on the fact that the capability for electrical cutting of tissue has been included in other devices, as illustrated in U.S. Pat. Nos. 5,941,876 and 7,674,263 B2, hereby incorporated by reference herein. All of these devices may suffer from the same fundamental limitations, the fact that the use of a lateral window may be an inherently inefficient and awkward means to cut tissue and these devices may therefore not be practical for removing any but the smallest lesions. As these devices may not be capable of longitudinal or axial cutting, such devices are limited in their ability to not only cut and remove larger and encapsulated lesions, but it may also be necessary to use another instrument to establish access to the lesion. Further such conventional systems do not possess the ability to longitudinally core into said lesion, and/or surrounding healthy tissue to produce a stratified cross-sectional sample of the lesion within said cutting needle which can be removed for later study and analysis. Additionally, such traditional devices are not capable of targeted delivery of therapeutic treatments into and/or around the lesion further limiting their usefulness.

The foregoing limitations associated with traditional surgical devices and therapeutic delivery systems as well as techniques of operation associated with said devices and systems may represent a long-felt need for a comprehensive, economical and effective solution to the same. While implementing elements may have been available, actual attempts to meet this need may have been lacking to some degree. This may have been due to a failure of those having ordinary skill in the art to fully appreciate or understand the nature of the problems and challenges involved. As a result of this lack of understanding, attempts to meet these long-felt needs may have failed to effectively solve one or more of the problems or challenges identified herein. These attempts may even have led away from the technical directions taken by the present inventive technology and may even result in the achievements of the present inventive technology being considered to some degree an unexpected result of the approach taken by some in the field.

Accordingly, there is a need within the field for an efficient and economically viable rotating needle surgical device and/or therapeutic delivery system that addresses each of the technological and economic limitations outlined above. The inventive technology disclosed in this application represents a significant leap forward within the field.

### DISCLOSURE OF INVENTIVE TECHNOLOGY

The present invention presents elements that can be implemented in various embodiments. Generally a goal of the present inventive technology is to provide, utilizing advancements in design, construction, assembly, materials, and other characteristics to provide a surgical device and therapeutic delivery system that is superior to devices and systems currently known in the art. These improvements will be taken up in detail as they are presented in the claims. Accordingly, the present invention includes a variety of aspects, which may be combined in different ways. The following descriptions are provided to list elements and describe some of the embodiments of the present invention. These elements are listed with initial and in some cases secondary or multiple embodiments, however it should be understood that they may be combined in any manner and in any number to create additional embodiments. The variously described examples and preferred embodiments should not be construed to limit the present invention to only the explicitly described systems, techniques, and applications. Further, this description should be understood to support and encompass descriptions and claims of all the various embodiments, systems, techniques, methods, devices, and applications with any number of the disclosed elements, with each element alone, and also with any and all various permutations and combinations of all elements in this or any subsequent application. Accordingly, the objects of the methods and apparatus for an improved rotating needle device and therapeutic treatment delivery system described herein address each of the foregoing in a practical manner.

One feature of the invention may be the ability to rotate a surgical device at ultra-high speeds minimizing the force required for axial insertion of, for example, a hollow cutting needle having a beveled cutting edge along its distal boundary and may even allow this cutting needle to pass through tissues of varying densities more easily. Another feature of the inventive technology may be the ability to rotate the surgical device at high speeds in the presence of a high magnetic field, which may make the invention capable of utilization in proximity of MRI systems. Another feature of the inventive technology may be the ability to remove and/or core a targeted tissue with a maximum of accuracy and perhaps even a minimum of manipulation and discomfort. Another feature of the inventive technology may be the ability to readily detect its position and even orientation under X-ray CT, MRI, and ultrasound guidance. Another feature of the inventive technology may be the ability to introduce a variety of therapeutic treatments directly into, or in the vicinity of the targeted lesion. Another feature of the inventive technology may be the ability to introduce other therapeutic treatments such as, but not limited to, probes, cryogenic devices, video devices, RF devices, lasers and other medical devices to the vicinity of the lesion. Another feature of the inventive technology may be the ability to longitudinally penetrate a target lesion capturing and preserving a stratified cross-sectional core tissue sample for removal and later analysis and testing. Another feature of the inventive technology may be the ability to efficiently remove and collect tissue pathologies into bio-hazard compliant elements utilizing an applied negative external pressure. Another feature of the inventive technology may be the ability to sterilize the majority of the components for additional re-use.

Naturally, further objects of the inventive technology will become apparent from the description and drawings below.

### BRIEF DESCRIPTION OF DRAWINGS

FIG. 1—is a blow-up perspective view of the distal leading boundary of a hollow cutting needle having a beveled edge in one embodiment thereof.

FIG. 2—is a cross-sectional view of a rotating needle device having a collection vessel attached to the proximal end of said device in one embodiment thereof.

FIG. 3—is a front view of a rotating needle device having a collection vessel attached to the proximal end of said device in one embodiment thereof.

FIG. 4A—is a front view of a therapeutic introducer in one embodiment thereof.

FIG. 4B—is a front view of a core collection cylinder having an internal separation membrane in one embodiment thereof.

FIG. 5—is a cross-sectional view of a therapeutic introducer having an internal delivery aperture and an o-ring groove and seal that may correspond to a hollow cutting needle in one embodiment thereof.

### MODE(S) FOR CARRYING OUT THE INVENTIVE TECHNOLOGY

As mentioned earlier, the present invention includes a variety of aspects, which may be combined in different ways. The following descriptions are provided to list elements and describe some of the embodiments of the present invention. These elements are listed with initial embodiments, however it should be understood that they may be combined in any manner and in any number to create additional embodiments. The variously described examples and preferred embodiments should not be construed to limit the present invention to only the explicitly described systems, techniques, and applications. Further, this description should be understood to support and encompass descriptions and claims of all the various embodiments, systems, techniques, methods, devices, and applications with any number of the disclosed elements, with each element alone, and also with any and all various permutations and combinations of all elements in this or any subsequent application. With all embodiments (whether methods and apparatus) that entail at least two element and/or steps in coordination, each association is contemplated to be direct and/or indirect as well as functional and or non-functional in nature. In addition, the term responsive, and/or responsive to may indicate that two elements may be coupled in a manner so as to be directly or indirectly connected. In further embodiments this may indicate that one element may respond with a discrete or non discrete action in response to the action or stimulus of a separate element.

As can be seen generally from figures, the invention consists of generic elements that may be embodied in many different forms. Certain embodiments of the current inventive technology describe a surgical device comprising: a hollow cutting needle (1) having a beveled cutting edge along its distal boundary (12) positioned within a rotatable support shaft (2) internally situated within a handle element (5); at least one fastener element (3) securing said cutting needle to said rotatable support shaft (2); at least one rotatable turbine (4) mechanically mated with said rotatable support shaft (2) and responsive to a non-electromagnetic-field generating power source (6); and at least one detachable collection vessel (7) responsive to an external negative pressure.

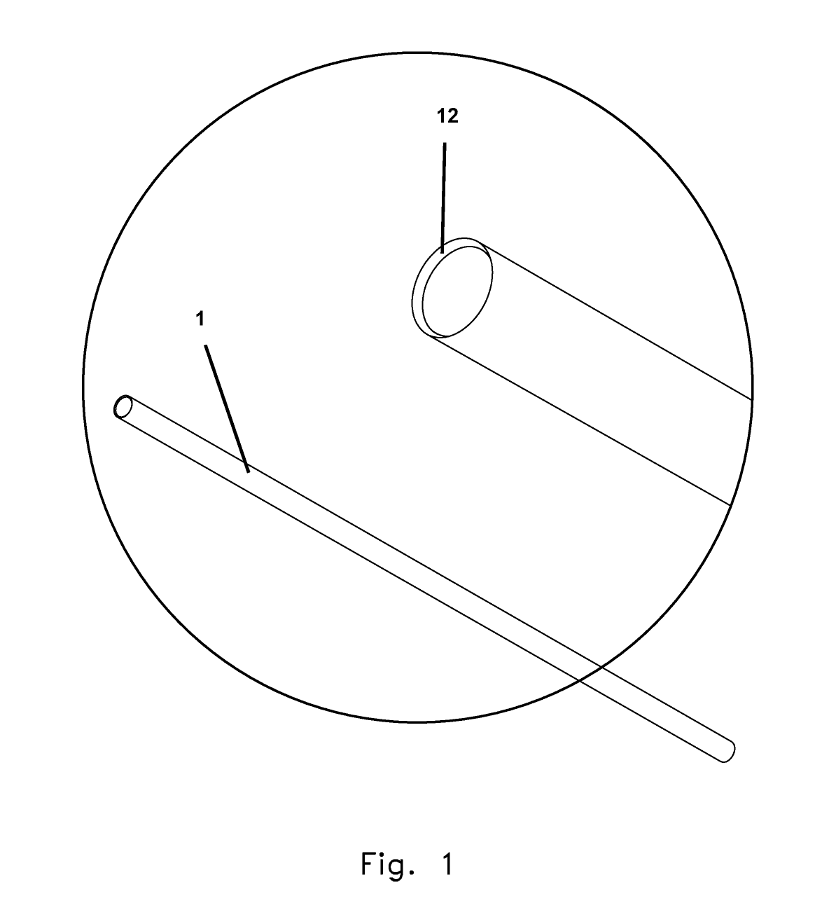

Now, viewing the various embodiments more specifically, as can be seen in FIG. 1, embodiments of the inventive surgical device may have a hollow cutting needle (1). Such a cutting needle may be formed from standard surgical grade steel, other appropriate metal(s) and/or other composite materials known with in the industry. As shown in the blown-up portion of figure, such a hollow cutting needle may present a variable sized lumen, perhaps with a diameter range of approximately 2 mm to 9 mm. Such lumen may be continuous, or perhaps non-continuous through the cutting needles resulting in only a partially hollow cutting needle. As detailed again in FIG. 1, to assist in the axial or longitudinal cutting of targeted tissue pathologies, a beveled cutting edge may be established along its distal boundary (12). Such a beveled edge may be further reinforced through composite material coatings such as graphite and or even a diamond-tipped coating. Further, such distal boundary may include a detachable distal boundary attachment based on a desired use having a variety of materials and/or shaped edges.

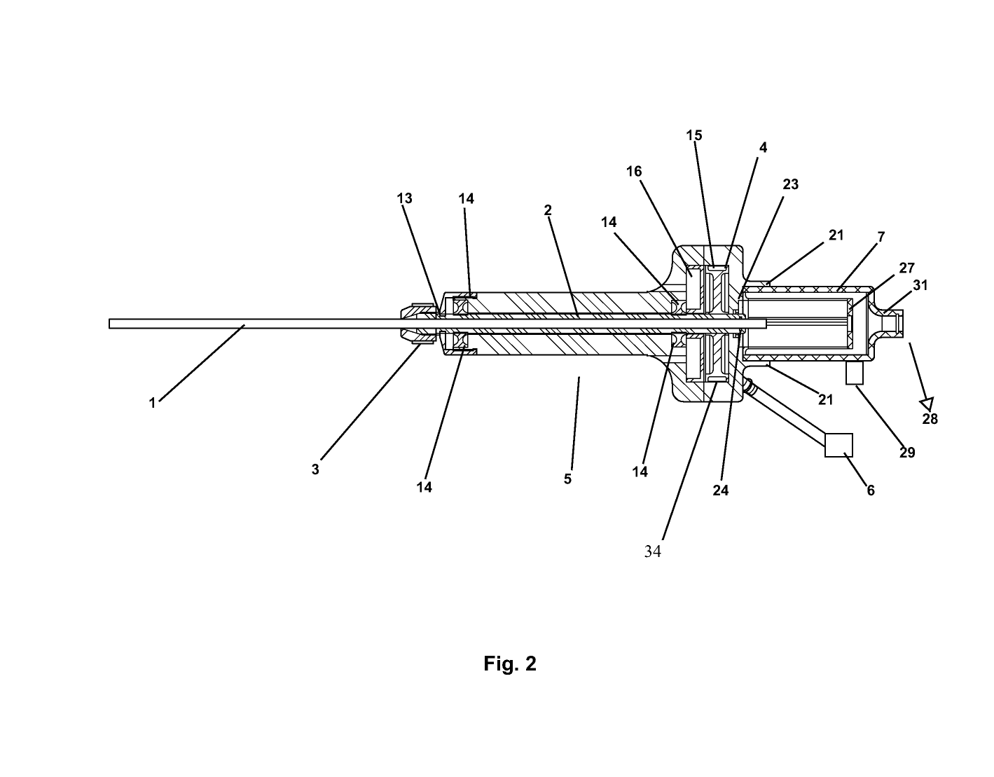

As described previously, a hollow cutting needle (1) having a beveled cutting edge along its distal boundary (12) may be manually positioned within a rotatable support shaft (2). Such rotatable support shaft (2) may be formed from any appropriate material such as hard-plastic, metal or other composite material, having a fitted position to accept the hollow cutting needle (1). As shown in FIG. 1, in certain embodiments a rotatable support shaft may have a distally tapered end-section which may act as an attachment point for a fastener element (3)—as will be shown below—to secure said hollow cutting needle (1) into the support shaft (2). In some embodiment this tapered end-section is circumferentially compressible in response to a fastener (3). This may be accomplished through perhaps spaced projection extending along the longitudinal axis of the support shaft (2). In this configuration, a fastener (3), such as a compression fastener may be placed over this compressible and/or tapered end-section compressing/securing the cutting needle (1) in place. In still another embodiment, a rotatable support shaft may have a slotted penetration extending outward from its body configured to accept a snap fastener. Such slotted snap fastener allows for the quick attachment and securing of a fastener (3) into a corresponding groove on the rotatable support shaft locking the cutting needle (1) within a rotatable support shaft (2). Such a slotted snap fastener may secure said cutting needle (1) through compression or physically blocking the needles forward movement. In still further embodiments, this tapered end-section may be threaded so as to accept a corresponding threaded fastener. As shown in FIG. 2, in one exemplary embodiment a cutting needle (1) is secured within a rotatable support shaft having a threaded tapered end by a threaded fastener, in this case a collet nut.

As can be appreciated, due to the high rotational force of the cutting needle (1), rotatable support shaft (2) and corresponding fastener, the invention may necessitate a system of easily disengaging said freely rotatable fastener element (3). As shown in FIG. 2, a preferred embodiment of the inventive technology may employ a slotted fastener leverage disengagement position (13). In a typical embodiment, a user may insert a blocking device, such as a wrench or other fitted tool to block rotation of said rotatable support shaft (2). Such a position may, in fact, represent a flattened or slotted position along the length of a rotatable support shaft (2) to accept such a blocking instrument. Having blocked the free rotation of said rotatable support shaft (2) a user may then disengage said fastener element. This embodiment being especially useful when employing a typical threaded fastener such as a collet nut.

Again referring generally to FIG. 2, as shown in some embodiments, a rotatable support shaft (2) may be positioned within a handle element (5) and supported by a plurality of internally positioned rotatable support shaft bearings (14). It should be noted that such bearings may be traditional metal rotatable bearings, or perhaps, as will be discussed in more detail below, to facilitate use of the aforementioned surgical device in the presence of certain active scanning devices such as MRI, CT, and the like, such bearings may be made of ceramic or other composite materials.

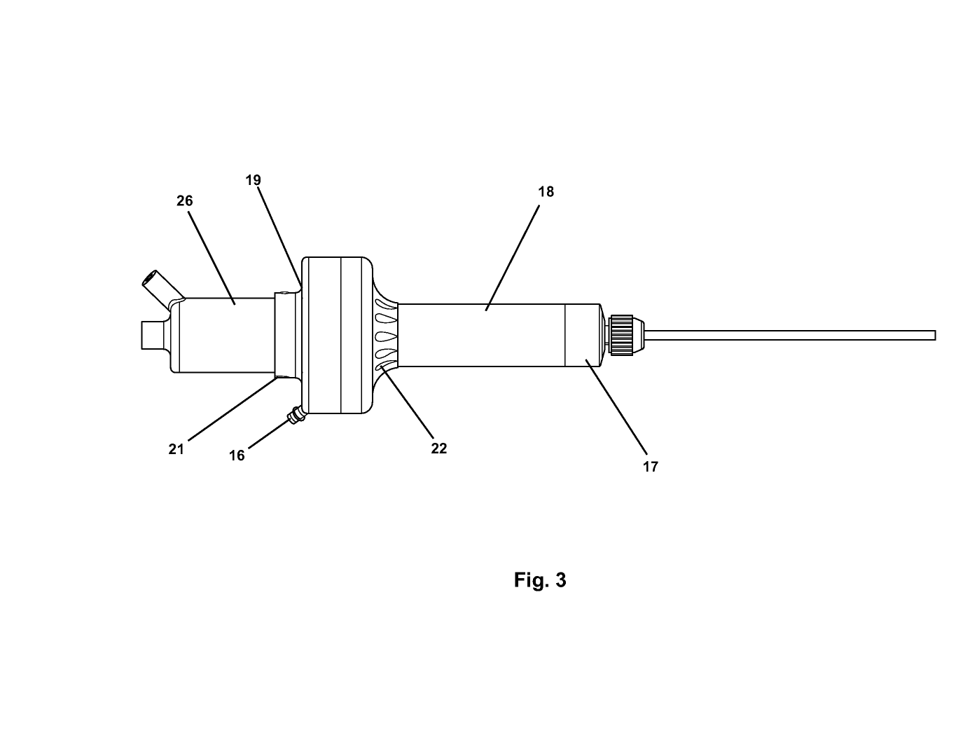

Referring back to FIGS. 2 and 3, in a preferred embodiment a handle element (5) may be comprised of a nose cap (17) attached to a grip element (18) which may further be secured to an end-cap (19) perhaps through a plurality of penetrating fasteners. These elements—generally forming the handle (5)—may be formed from hard-plastic or other composite materials. In addition, such elements may be individually shaped through molding, casting, mold-injection, and/or 3-D printing techniques generally known in the art. Now, as shown specifically in FIG. 2, the association of the end-cap (19) and grip element (18) may form an internal turbine cavity (15). Further, the nose cap (17), in certain embodiments may be exemplified by a snap fitting nose cap, a lock fitting nose cap; and/or slide fitting nose cap for quick attachment and/or removal.

Again returning to FIG. 2, as discussed above, the association of a grip element (18) with said end-cap (19) forms an internal turbine cavity wherein may be positioned a rotatable turbine (4) which may further be mechanically mated with said rotatable support shaft (2). Such a rotatable turbine (4) may be internally supported by a central bearing mechanism, which as before, may be formed from ceramic or other composite materials. Such rotatable turbine (4) may further be constructed from a variety of materials including, ceramics, composites, hard-plastics and/or metals. In a preferred embodiment all such material would be sterilizeable from one-procedure to the next.

Now, as shown in FIGS. 2 and 3, as stated above, in this embodiment the rotatable turbine (4) is responsive to a non-electromagnetic-field generating power source (6) (and by implication the rotatable support shaft (2) securing a cutting needle (1) being mechanically mated with the rotatable turbine (4) is responsive to a non-electromagnetic-field generating power source (6)). Of course, any such power source may be contemplated in the various embodiments of the inventive technology, including electrical, and/or mechanical power sources. However, as pointed out previously, it is extremely advantageous to operate the current invention in the presence of active and X-ray scan, and/or active CT scan, and/or active MRI scan, and/or active ultrasound scan without causing the creation of an electro-magnetic field which would create interference in the operation of said scans. In this case, such non-electromagnetic-field generating power source (6) may include any power source that does not generate an electro-magnetic field. In a preferred embodiment such non-electromagnetic-field generating power source (6) may include a pneumatic power source and/or a hydraulic power source.

As show again in FIGS. 2 and 3, the invention may have a handle element containing an integral external power source access position (16). Such an element may be integral to the end-cap (19). As shown in FIG. 3, such integral external power source access position (16), in the case of a pneumatic power source, may include an air intake position. As can easily be appreciated, in order to rotate the turbine at a high rate of speed (up to as much as 90,000 RPM, if so desired) compressed air from a pneumatic power source may pass through an air intake position causing a turbine, and associated elements to rotate at a regulated speed. (Such speed being regulated by the inflow of air from a pneumatic power source control element, which many be automatic, or computer directed and/or manually operable by a user.) As shown in FIG. 2, in the currently described embodiment, to more efficiently capture a non-electromagnetic-field generating power source (6), such as compressed air from a pneumatic power source, in some embodiments the rotatable turbine (4) may have a plurality of turbine blade positions (34). These turbine blade positions (34) may be placed so that, for example, an air intake position is configured substantially tangential to said rotatable turbine so as to exert force on these turbine blade positions (34) optimizing energy transfer and rotation.

As indicated earlier, the inventive technology described herein may achieve rotational velocities of approximately 90,000 RPM if so desired. While this RPM may be controlled through manipulation of a non-electromagnetic-field power source control element, such high velocities, coupled with the enhanced axial cutting ability facilitated by the hollow cutting needle (1) having a beveled cutting edge along its distal boundary (12), it may be advantageous to quickly and efficiently cut and/or core various types and densities of tissue pathologies. However, as can be expected such rotational velocities and air displacement may cause a significant sound component. As such, in a preferred embodiment the inventive technology may have a turbine muffler (16). As shown in FIG. 2, the coordination of said grip element (18) and end-cap (19) may form an internal turbine muffler cavity. In certain embodiment such a turbine muffler (16) may include at least one integral convoluted pathway directing, and/or redirecting air-flow so as to diffuse the vibrational sound waves. Such integral convoluted pathway may be coupled with, for example, a plurality of exhaust ports. Such exhaust ports begin in some case integral or continuous with said grip element. Again referring to FIG. 2, in a preferred embodiment said turbine muffler (16) may include a material insert, such as a nylon mesh insert to muffle, diffuse and/or redirect excessive sound vibration.

As can naturally be appreciated, during operation of the surgical device, for example to remove a tissue pathology representing bio-hazardous material, an appropriate collection element may be employed to collect the extracted tissue in a safe and efficient manner. As shown generally in FIGS. 2 and 3, a preferred embodiment of the current inventive technology may have at least one detachable collection vessel (7) responsive an external negative pressure such as a vacuum. As can naturally be assumed, such vacuum may be an automatic motor generated vacuum and/or a manually generated vacuum perhaps through operation of a syringe having a retractable plunger element attached to an external negative pressure port (28) or vacuum port located, for example, on the posterior aspect of the detachable collection vessel (7). In a preferred embodiment, such collection vessel (7) may be secured to an extended slide lock position (21) on the end-cap (19) by the insertion of a slide lock projection (36) into a slide lock position (21).

As can naturally be appreciated, application of external negative pressure may cause an upward vacuum pressure to be exerted through not only the detachable collection vessel (7), but the hollow cutting needle (1) itself. As shown in FIG. 2, this hollow cutting needle (1) may partially penetrate into the detachable collection vessel (7) through a sealed cutting needle insert aperture. This sealed cutting needle insert aperture may be sealed by securement of a cutting needle seal (24) fitted to the outer circumference of the cutting needle (1). In one embodiment this seal may be an o-ring and groove seal. Additionally, a vacuum seal (25) may be internally placed around the rotatable support shaft (2) to maintain the vacuum integrity between the collection vessel (7), cutting needle (1), rotatable support shaft (2) and internal turbine cavity (15). Finally, in a preferred embodiment, a seal retainer plate (23) may be internally positioned to be placed flush with a collection vessel (7), or a fluid barrier (27) as will be discuss below, to further maintain the separation and vacuum integrity between the rotatable support shaft (2) and the internal turbine cavity (15), as well as reducing the risk of tissue contamination throughout the system. In this manner, a vacuum force may be externally created and then applied through the external negative pressure port (28), through a detachable collection vessel (7), hollow cutting needle (1) and ultimately on the targeted tissue to be cut, sampled, removed and/or cored.

Again generally referring to FIG. 2, as can be appreciated in some embodiments the upward movement and eventual expulsion of tissue and fluid from the target pathology may, if not prevented from doing so, continue along through to the source of the vacuum force. In certain embodiments, such tissue removal and extraction may be desired as such an external negative pressure may be generated by an appropriate device capable of accepting and separating waste tissue and fluid from the larger apparatus. Such bio-hazard vacuum devices are known in the art and can be easily adapted to the following device.

However, in further embodiments it may be desired to retain the extracted tissue pathology for appropriate disposal and/or study. In this case, some embodiments of the inventive technology may have a detachable collection vessel (7) having a filter barrier (27) internally placed inside said collection vessel (7) and around said cutting needle (1). In a preferred embodiment, tissue and fluid extracted from the target pathology travels up the lumen of the cutting needle (1) and is initially deposited into a filter barrier (27). Such a filter barrier may retain only solid/semi-solid material such as tissue, while allowing fluids, such as irrigation fluid or other bio-fluids to pass through the filter into the collection vessel (7). These filtered fluids may further be removed from the collection vessel (7) through a fluid port (29), perhaps by application of a second external negative pressure, or even simple draining action as dictated by the fluid port's placement and gravity. As can further be appreciated, it may be desirable to prevent any fluid flow through the external negative pressure port. As such, certain embodiments of the current invention may include a collection vessel (7) configured with a separation membrane (31) preventing any fluid flow through said port. As such, this separation membrane (31) creates a closed system reducing bio-waste, as well as the amount of material that will need post-procedure sterilization processing.

It should be specifically noted that the aforementioned elements—forming the broad surgical device herein described—may be designed to be operable in the presence of an active X-ray scan, and/or active CT scan, and/or active MRI scan, and/or active ultrasound scan. For example, traditional lateral cutting devices cannot be effectively real-time imaged using ultrasound scans, as there are insufficient vibrational and/or reflective surfaces capable to visualizing such traditional devices. In most instances, for example, an ultrasound may need to be positioned nearly perfectly perpendicular to such a traditional device to be seen at all. However, the current inventive technology may be seen by ultra-sound techniques even while not positioned perpendicular to the cutting needle as a result of the vibrational output coupled with the novel axial cutting configuration. Indeed, such active scans may be used in conjunction with this surgical device to generate real-time or approximate real-time imaging of a targeted pathology. Such scans may be used to guide the cutting needle to the target tissue, as well as produce real-time or approximately real-time images of the removal and/or coring of the target tissue pathology enhancing the therapeutic capabilities of such an invention.

As can be appreciated from the foregoing recitation, in a preferred embodiment the above described surgical device may be utilized to sample and/or remove an identified lesion. In a preferred embodiment, for example, tissue pathology may be identified, perhaps visually, or through a variety of imagining systems such as, but not limited to X-ray, and/or CT, and/or MRI, and/or active ultrasound scans. In this instance a user of the above described surgical device may secure a selected hollow cutting needle having a beveled cutting edge along its distal boundary within a rotatable support shaft internally situated within a handle element. This step may be accomplished manually on a per-use basis by a user according to, for example, a desired length, distal cutting surface, lumen diameter and the like, or may (as with all assembly type steps) be pre-fabricated by a manufacture. Next, a user may fasten said cutting needle to said rotatable support shaft. Next a user may insert a guide channel directed to the tissue pathology to be removed. In some instances this guide channel (not shown) is a hollow tube of a variable length that is inserted in an appropriate incision on, for example, a patient. Such a guide channel may act to bypass non targeted tissues and provide a direct line for the cutting needle to follow. In some instances the step of inserting a guide channel directed to the tissue pathology to be removed comprises the step of inserting a solid introducer rod having a diameter smaller that the lumen of said hollow cutting needle directed to the tissue pathology to be removed. In this embodiment, a user may insert said hollow cutting needle over the previously inserted solid rod directed to the tissue pathology to be removed so as to allow said rod to pass through the lumen of said cutting needle directing the cutting needle to the target tissue.

At this point, a user may initiate the generation of an external negative pressure creating a vacuum force through said cutting needle then insert said cutting needle through said guide channel (or around a solid introducer rod in an alternative embodiment). The user, having placed the cutting needle in the appropriate position—perhaps guided by the imaging assistance provided by an active X-ray, and/or CT, and/or MRI, and/or active ultrasound scans—may actuate the rotatable turbine mechanically mated with said rotatable support shaft through application of a non-electromagnetic-field generating power source on said rotatable turbine. This step, as can be deduced rotates the cutting needle to a desire RPM allowing the user to penetrate the target tissue pathology with said cutting needle to a desired depth, again, guided by visualization assistance provided by an active X-ray, and/or CT, and/or MRI, and/or active ultrasound scans in some embodiments. As the hollow cutting needle penetrates the tissues the vacuum extracts the severed tissue pathology upwardly though the hollow cutting needle depositing the extracted tissue into at least one detachable collection vessel. After the targeted tissue has been a removed or otherwise treated to the user's satisfaction, the non-electromagnetic-field generating power source may be deactivated through a control element stopping rotation of said turbine and consequently the associated cutting needle may be retracted.

In further embodiments, a user may detach the collection vessel containing said extracted tissue for disposal and/or further study, as well as loosen the fastener and remove said cutting needle for disposal. Further, as an economic benefit, the remaining handle and internal components can be easily broken down and sterilized for re-use. Such sterilization procedures may include, but are not limited to, sonication sterilization; autoclave sterilization; gas sterilizing; chemical sterilization; heat sterilization; and enzymatic sterilization and the like.

As previously discussed, the current inventive technology utilizes a novel hollow cutting needle having a beveled cutting edge along its distal boundary, coupled with the ability to generate extremely high RPM. This allows for the unique and novel axial/longitudinal penetration of targeted pathologies. Such tissue “cores” may be axially sheared such that the target tissue is substantially intact and minimally damaged. In some instances, it may be advantageous to extract a whole or substantially whole cross section of said tissue pathology. Such cross-section may be stratified, such that each distinct layer of cellular material is visible and/or identifiable. Further, such “core” sections may include layers of tissue containing the diseased pathology in addition to healthy tissues allowing for evaluation and comparison. It can plainly be seen that such “tissue coring” of stratified cross sections of tissue pathologies, or hybrid pathologies and healthy tissue can serve as a valuable diagnostic tool allowing for differential analysis of the various stratified layers.

As such, certain embodiment of the current technology may encompass a surgical device for tissue coring comprising a hollow cutting needle (1) having a beveled cutting edge along its distal boundary (12) positioned within a rotatable support shaft (2) internally situated within a handle element (5); at least one fastener element (3) securing said cutting needle to said rotatable support shaft (2); at least one rotatable turbine (4) mechanically mated with said rotatable support shaft (2) and responsive to a non-electromagnetic-field generating power source (6); and at least one core collection cylinder (30) contiguous with said cutting needle and responsive an external negative pressure.

In some embodiments such a core collection cylinder (30) maybe removable, such that it may be inserted into an already positioned detachable collection vessel (26) and sealed to the proximal end of cutting needle to accept an extracted tissue core. In still further embodiments, a core collection cylinder (30) may be fitted to the end of said hollow cutting needle (1) post-extraction—with the accumulated stratified cross-sectional sample of tissue intact within the hollow needle—to accept an extracted tissue core in response to a positive pressure applied to the hollow cutting needle. Such positive pressure may be applied to the opposite end of the cutting needle, perhaps by reversing the flow of the external pressure from negative to positive. Or, in another embodiment, a simple syringe may be placed on the opposing end from a fitted core collection cylinder (30) and through action of said syringe plunger extract said stratified cross-sectional sample of tissue into the core collection cylinder (30).

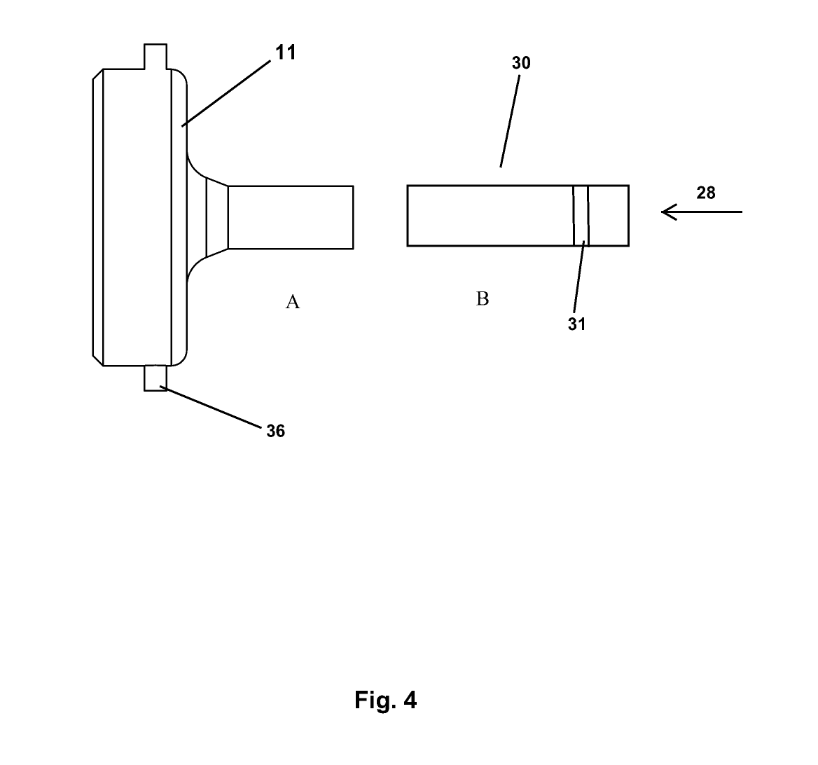

As generally shown in FIG. 4B, in a preferred embodiment a core collection cylinder (30) may be an elongated hollow tube, perhaps made of plastic or other appropriate material that may act as a receptacle for biological tissue. In addition such, device would preferably be sterile, or sterilizeable, as well as have the ability to be frozen, for example, in liquid nitrogen for long term biological storage. In some embodiments the core collection cylinder (30) may be contiguous with a cutting needle, such that it is able to attach to the end of said cutting needle (1) forming a continuous lumen to accept an tissue “core,” perhaps through a cutting needle sealed insert aperture. In some embodiments a core collection cylinder (30) may be continuously sealed to a cutting needle (1) through an o-ring and groove seal securable to said hollow cutter needle. (It should be noted that a cutting needle (1) having a compatible groove for an o-ring and groove seal is contemplated within this invention.) Additional embodiments may contemplate a clamp attachment or even a slide lock projection, which may also be designed to be compatible with the slotted slide lock (21) on the device's end-cap (19). Further, the core collection cylinder (30) may also be responsive to an external negative pressure, such that it may have at least one external negative pressure port (28) responsive to an external negative pressure generator, whether that be mechanical and/or manual. (not shown).

To address the concerns noted above regarding control and containment of any extracted tissue and/or fluid samples, certain embodiments of the invention may include a core collection cylinder having at least one filter barrier (not shown), as well as a fluid port (not shown), as well as at least one separation membrane (31) to prevent any cored tissue from inadvertently entering the external port, as well as providing a barrier during extraction of said stratified cross-sectional sample of tissue.

As can be appreciated from the foregoing recitation, in a preferred embodiment the above describe surgical device may be utilized in a method of tissue coring using a rotating needle device. Such a method may include, for example: securing a hollow cutting needle having a beveled cutting edge along its distal boundary within a rotatable support shaft internally situated within a handle element; fastening said cutting needle to said rotatable support shaft; inserting a guide channel directed to the tissue to be cored; guiding said cutting needle through said guide channel to said tissue to be cored; actuating a rotatable turbine mechanically mated with said rotatable support shaft through application of a non-electromagnetic-field generating power source on said rotatable turbine; axially or longitudinally penetrating said tissue with said cutting needle to a desired depth, which may be assisted by visual imaging provided by active an active X-ray scan, and/or active CT scan, and/or active MRI scan, and/or active ultrasound scan; and accumulating a stratified cross-sectional sample of tissue within said cutting needle through the action of the upward vacuum force exerted on the extracted target tissue; deactivating said non-electromagnetic-field generating power source stopping rotation of said turbine with the stratified cross-sectional sample of tissue still within the needle; attaching a core collection cylinder contiguous with said cutting needle; and extracting said stratified cross-sectional sample of tissue from said cutting needle into said core collection cylinder by application of an external negative pressure. In some embodiments the external negative pressure will extract the stratified cross-sectional sample of tissue from said cutting needle into said core collection cylinder prior to stopping rotation of the cutting needle.

In some embodiments the external negative pressure will extract the stratified cross-sectional sample of tissue from said cutting needle into said core collection cylinder prior to stopping rotation of the cutting needle. It should also be noted that the aforementioned system may be configured so as to be operable, and even assisted by in the presence of an active X-ray scan, and/or active CT scan, and/or active MRI scan, and/or active ultrasound scan without causing interference in said scans. Such operability and assistance may be in real, or approximate real-time.

Still further embodiments of the current inventive technology contemplate a method of tissue coring using a surgical device comprising the steps of: securing a hollow cutting needle having a beveled cutting edge along its distal boundary within a rotatable support shaft internally situated within a handle element; fastening said cutting needle to said rotatable support shaft; fastening a core collection cylinder responsive to an external negative pressure to said cutting needle; inserting a guide channel directed to the tissue to be cored; activating said external negative pressure creating a vacuum within said cutting needle; actuating a rotatable turbine mechanically mated with said rotatable support shaft through application of a non-electromagnetic-field generating power source on said rotatable turbine; guiding said cutting needle through said guide channel; axially longitudinally penetrating said tissue with said cutting needle to a desired depth and removing a stratified cross-sectional sample of tissue within said cutting needle; accumulating said stratified cross-sectional sample of tissue within said core collection cylinder through action of said external negative pressure; de-activating said external negative pressure; deactivating said non-electromagnetic-field generating power source stopping rotation of said turbine; and extracting said stratified cross-sectional sample of tissue from said core collection cylinder.

Again, it should be noted that the aforementioned system may be configured so as to be operable, and even visually assisted by the presence of an active X-ray scan, and/or active CT scan, and/or active MRI scan, and/or active ultrasound scan without causing interference in said scans. Such operability and assistance may be in real, or approximate real-time.

Generally referring to FIGS. 2-5, certain embodiments of the current inventive technology contemplate device and/or system for delivering in situ therapeutic treatment. In a preferred embodiment such a system may comprise a rotating needle device comprising a hollow cutting needle (1) having a beveled cutting edge along its distal boundary (12) positioned within a rotatable support shaft (2) internally situated within a handle element (5); at least one fastener element (3) securing said cutting needle to said rotatable support shaft (2); at least one rotatable turbine (4) mechanically mated with said rotatable support shaft (2) and responsive to a non-electromagnetic-field generating power source (6); at least one detachable therapeutic introducer (11) having a delivery aperture (10) fitted to the posterior aspect of said cutting needle; and at least one therapeutic treatment insert (not shown) configured to pass into said delivery aperture and through said cutting needle so as to deposit said therapeutic treatment in situ.

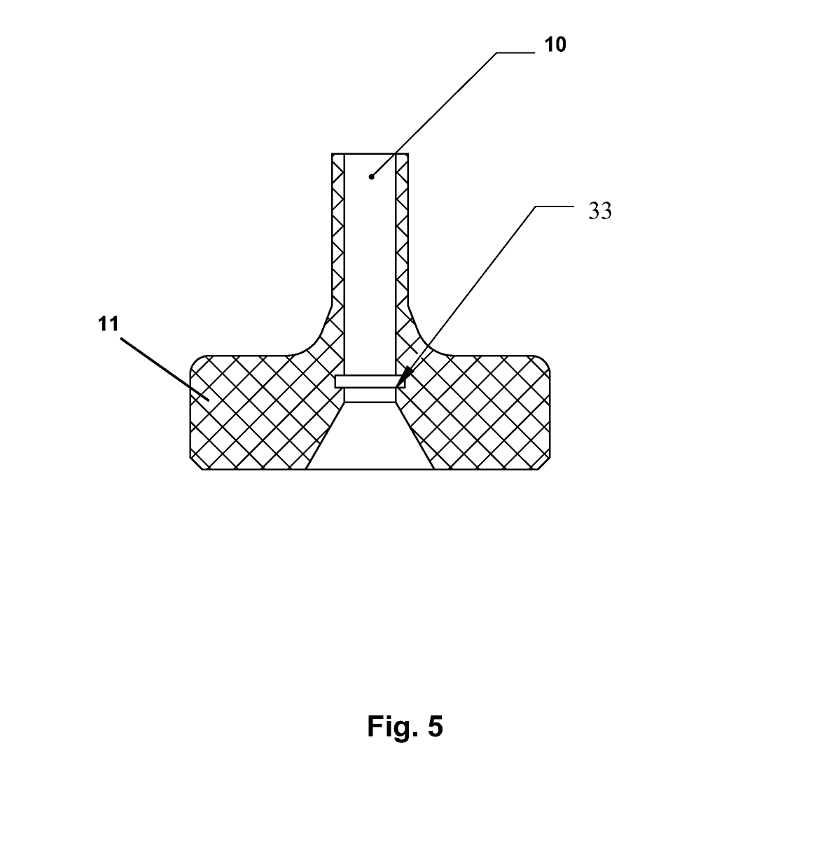

Referring specifically to FIGS. 4A and 5, such a therapeutic introducer (11) may have, in some embodiments, at least one slide lock projection (36) allowing it to be attached and detached to the slotted slide lock element(s) (21) on the device end cap (19). In this manner, it is possible, as will be shown, to remove a targeted tissue pathology utilizing a detachable collection vessel (7) which can subsequently be removed, and replaced with a therapeutic introducer (11) to facilitate the introduction of a therapeutic treatment in situ. Again referring to FIG. 5, certain embodiments may contemplate a detachable therapeutic introducer (11) having an o-ring and groove seal (33) which may be fitted over the proximal end of the hollow cutting needle (1) forming a seal.

As can be seen from this configuration, after attachment of a therapeutic introducer (11) to the hollow cutting needle (1) a direct sealed pathway has been created to the target tissue or the void where the target tissue was previously situated. In this configuration, a user may introduce a therapeutic treatment insert (not shown), down the pathway to the target tissue, automatically assisted by a pressurized machine dispenser or perhaps manually with a simple pressurized device such as a syringe. It should be noted that the term therapeutic treatment may be construed broadly to include any medical treatment, application, and/or diagnostic application. In certain embodiments, such therapeutic treatment(s) may include, any: 1) therapeutic liquid, 2) therapeutic solid; 3) therapeutic probe; and 4) therapeutic gas. Examples of such therapeutic treatments may include by way of specific example: irrigation fluid, anesthetic, cryo-probe; heat probe; laser probe; radioactive element; radioactive solution; chemotherapeutic agent; antigenic agent; pharmaceutical; cellular deposit; stem-cell deposit; stain; immunosuppressant; immuno-activator; anti-inflammatory; steroid; adjuvant; anti-biotic; growth factor; hormone; RF ablation element; biological marker; chemical marker; artificial material; buffer solution; and alcohol. It should be noted that such a list is merely representative and in no way limiting of the variety of therapeutic treatments broadly contemplated in the invention.

Additional embodiments also contemplate apparatus and methods for substantially removing said in situ therapeutic treatment. In a preferred embodiment, removal of a therapeutic treatment, such as a liquid, may be accomplished by the step of generating an external negative pressure creating a vacuum force through said cutting needle substantially removing the in situ therapeutic treatment.

It should also be noted that the aforementioned system may be configured so that delivery, deposition, concentration, penetration, distribution of said therapeutic treatment insert is configured to be operable, and even assisted by the presence of an active X-ray scan, and/or active CT scan, and/or active MRI scan, and/or active ultrasound scan without causing interference in said scans. Such operability and assistance may be in real, or approximate real-time. Further, it should be noted that the aforementioned system may be configured so that delivery, deposition, concentration, penetration, distribution and even effect of said therapeutic treatment insert is configured to be operable, and even assisted by the presence of an active X-ray scan, and/or active CT scan, and/or active MRI scan, and/or active ultrasound scan or even video without causing interference in said scans. Such operability and assistance may be in real, or approximate real-time.

Finally, the following clauses have been presented as part of the specification and herein describe embodiments the inventive technology broadly as:
1. A surgical device comprising:
   a hollow cutting needle having a beveled cutting edge along its distal boundary positioned within a rotatable support shaft internally situated within a handle element;
   at least one fastener element securing said cutting needle to said rotatable support shaft;
   at least one rotatable turbine mechanically mated with said rotatable support shaft and responsive to a non-electromagnetic-field generating power source; and
   at least one detachable collection vessel responsive an external negative pressure.
2. A surgical device as described in clause 1 or any other clause wherein said hollow cutting needle having a beveled cutting edge along its distal boundary comprises a hollow cutting needle having a serrated cutting edge along its distal boundary.
3. A surgical device as described in clause 1 or any other clause wherein said hollow cutting needle having a beveled cutting edge along its distal boundary comprises a hollow cutting needle having a blunt distal cap with split lateral apertures along its distal boundary to facilitate tissue side harvesting.
4. A surgical device as described in clause 1 or any other clause wherein said hollow cutting needle having a beveled cutting edge along its distal boundary comprises a hollow cutting needle having a beveled cutting edge along its distal boundary with a lumen diameter range of approximately 2 mm to 9 mm.
5. A surgical device as described in clause 1 or any other clause wherein said rotatable support shaft comprises a rotatable support shaft having a distally tapered end-section.
6. A surgical device as described in clause 5 or any other clause wherein said rotatable support shaft having a distally tapered end-section comprises a rotatable support shaft having a distally tapered end-section where said tapered end-section is circumferentially compressible.
7. A surgical device as described in clause 5 or any other clause wherein said rotatable support shaft having a distally tapered end-section comprises a rotatable support shaft having a distally tapered end-section where said tapered end-section is threaded so as to accept a corresponding fastener.
8. A surgical device as described in clause 1 or any other clause wherein said rotatable support shaft comprises a rotatable support shaft having a slotted penetration to accept a snap fastener.
9. A surgical device as described in clause 1 or any other clause wherein said rotatable support shaft comprises a rotatable support shaft having a slotted fastener leverage disengagement position.
10. A surgical device as described in clause 1 or any other clause wherein said fastener element securing said cutting needle to said rotatable support shaft comprises a compression fastener.
11. A surgical device as described in clause 1 or any other clause wherein said fastener element securing said cutting needle to said rotatable support shaft comprises a slotted snap fastener.
12. A surgical device as described in clause 1 or any other clause wherein said fastener element securing said cutting needle to said rotatable support shaft comprises a threaded fastener.
13. A surgical device as described in clause 12 or any other clause wherein said threaded fastener comprises a collet nut.
14. A surgical device as described in clause 1 or any other clause and further comprising a plurality of rotatable support shaft bearings.
15. A surgical device as described in clause 14 or any other clause wherein said plurality of rotatable support shaft bearings comprises a plurality of ceramic rotatable support shaft bearings.
16. A surgical device as described in clause 1 or any other clause wherein said handle element comprises at least one grip element coordinated with at least one end cap and at least one nose cap forming an internal turbine cavity.
17. A surgical device as described in clause 16 or any other clause wherein said nose cap comprises a nose cap selected from the group consisting of: a snap fitting nose cap, a lock fitting nose cap; and slide fitting nose cap.
18. A surgical device as described in clause 16 or any other clause wherein said grip element is secured to said end cap by a plurality of penetrating fasteners.
19. A surgical device as described in clause 16 or any other clause wherein said end cap comprises at least one detachable collection vessel slide lock position.
20. A surgical device as described in clause 1 or any other clause wherein said handle element comprises an integral external power source access position.
21. A surgical device as described in clause 20 or any other clause wherein said integral external power source access position comprises at least one air intake position.
22. A surgical device as described in clause 21 or any other clause wherein said air intake position comprises an air intake positioned substantially tangential to said rotatable turbine.
23. A surgical device as described in clause 1 or any other clause and further comprising at least one turbine muffler.
24. A surgical device as described in clause 23 or any other clause wherein said turbine muffler comprises at least one integral convoluted pathway.
25. A surgical device as described in clause 23 or any other clause wherein said turbine muffler comprises at least one nylon mesh insert.
26. A surgical device as described in clause 1 or any other clause wherein said handle element comprises a handle element having plurality of exhaust ports.
27. A surgical device as described in clause 1 or any other clause wherein said rotatable turbine comprises a rotatable turbine having plurality of turbine blade positions.
28. A surgical device as described in clause 1 or any other clause and further comprising at least one turbine bearing supporting said rotatable turbine.
29. A surgical device as described in clause 28 or any other clause wherein said turbine bearing comprises a ceramic turbine bearing.
30. A surgical device as described in clause 1 or any other clause wherein said rotatable turbine comprises a plastic rotatable turbine.
31. A surgical device as described in clause 1 or any other clause and further comprising at least one seal retainer plate.
32. A surgical device as described in clause 1 or any other clause and further comprising at least one cutting needle seal.
33. A surgical device as described in clause 32 or any other clause wherein said cutting needle seal comprises an o-ring and groove seal.
34. A surgical device as described in clause 1 or any other clause and further comprising at least one vacuum seal.
35. A surgical device as described in clause 1 or any other clause wherein said non-electromagnetic-field generating power source comprises a non-electromagnetic-field generating power source selected from the group consisting of: a pneumatic power source; and a hydraulic power source.
36. A surgical device as described in clause 1 or any other clause wherein said detachable collection vessel comprises a detachable collection vessel having a sealed cutting needle insert aperture.
37. A surgical device as described in clause 1 or any other clause wherein said detachable collection vessel comprises a detachable collection vessel having a fluid port.
38. A surgical device as described in clause 1 or any other clause wherein said detachable collection vessel comprises a detachable collection vessel having filter barrier.
39. A surgical device as described in clause 1 or any other clause wherein said detachable collection vessel comprises a detachable collection vessel having an external negative pressure port.
40. A surgical device as described in clause 39 or any other clause wherein said external negative pressure port comprises a sealed vacuum port.
41. A surgical device as described in clause 1 or any other clause wherein said detachable collection vessel comprises a detachable collection vessel having separation membrane.
42. A surgical device as described in clause 1 or any other clause wherein said detachable collection vessel comprises a detachable collection vessel having at least one slide lock projection.
43. A surgical device as described in clause 1 or any other clause wherein said external negative pressure comprises a vacuum.
44. A surgical device as described in clause 43 or any other clause wherein said vacuum comprises an automatic motor generated vacuum and/or a manually generated vacuum.
45. A surgical device as described in clause 44 or any other clause wherein said manually generated vacuum comprises a manually generated vacuum through operation of a syringe having a retractable plunger element.
46. A surgical device as described in clause 1 or any other clause and further comprising at least one guide channel.
47. A surgical device as described in clause 46 or any other clause wherein said guide channel comprises a solid introducer rod having a diameter smaller that the lumen of said hollow cutting needle.
48. A surgical device as described in clause 1 or any other clause wherein said surgical device comprises a surgical device operable in the presence of an active X-ray scan, and/or active CT scan, and/or active MRI scan, and/or active ultrasound scan without causing interference in said scans.
49. A surgical device as described in clause 1 or any other clause and further comprising a non-electromagnetic-field generating power source control element.
50. A surgical device as described in clause 1 or any other clause and further comprising an external negative pressure control element.
51. A method of removing tissue pathologies utilizing a surgical device comprising the steps of:
   securing a hollow cutting needle having a beveled cutting edge along its distal boundary within a rotatable support shaft internally situated within a handle element;
   fastening said cutting needle to said rotatable support shaft;
   inserting a guide channel directed to the tissue pathology to be removed;
   generating an external negative pressure creating a vacuum force through said cutting needle;
   inserting said cutting needle through said guide channel;
   actuating a rotatable turbine mechanically mated with said rotatable support shaft through application of a non-electromagnetic-field generating power source on said rotatable turbine;
   penetrating said tissue pathology with said cutting needle to a desired depth;
   extracting said tissue through application of said vacuum force through said cutting needle;
   depositing said extracted tissue pathology into at least one detachable collection vessel;
   deactivating said non-electromagnetic-field generating power source stopping rotation of said turbine; and
   retracting said cutting needle.
52. A method of removing tissue pathologies utilizing a surgical device as described in clause 51 or any other clause and further comprising the step of loosening said fastener and removing said cutting needle.
53. A method of removing tissue pathologies utilizing a surgical device as described in clause 51 or any other clause and further comprising the step of detaching said collection vessel containing said extracted tissue.
54. A method of removing tissue pathologies utilizing a surgical device as described in clause 51 or any other clause and further comprising the step of sterilizing said handle and internal components for re-use.
55. A method of removing tissue pathologies utilizing a surgical device as described in clause 54 or any other clause wherein said step of sterilizing said handle and internal components for re-use comprises the step of sterilizing selected from the group consisting of: sonication sterilization; autoclave sterilization; gas sterilization; chemical sterilization; heat sterilization; and enzymatic sterilization.
56. A method of removing tissue pathologies utilizing a surgical device as described in clause 51 or any other clause wherein said step of inserting said cutting needle through said guide channel comprises the step of inserting said cutting needle through said guide channel in the presence of an active X-ray scan, and/or active CT scan, and/or active MRI scan, and/or active ultrasound scan without causing interference in said scans.
57. A method of removing tissue pathologies utilizing a surgical device as described in clause 51 or any other clause wherein said step of penetrating said tissue pathology with said cutting needle to a desired depth comprises the step of utilizing the visual assistance of an active X-ray scan, and/or active CT scan, and/or active MRI scan, and/or active ultrasound scan to guide said tissue penetration.
58. A method of removing tissue pathologies utilizing a surgical device as described in clause 57 or any other clause wherein said scan guided tissue penetration is approximately real-time.
59. A method of removing tissue pathologies utilizing a surgical device as described in clause 51 or any other clause wherein said step of extracting said tissue through application of said vacuum force through said cutting needle comprises the step of extracting said tissue through application of said vacuum force through said cutting needle in the presence of an active X-ray scan, and/or active CT scan, and/or active MRI scan, and/or active ultrasound scan without causing interference in said scans.
60. A method of removing tissue pathologies utilizing a surgical device as described in clause 51 or any other clause wherein said step of extracting said tissue through application of said vacuum force through said cutting needle comprises the step of utilizing the visual assistance of an active X-ray scan, and/or active CT scan, and/or active MRI scan, and/or active ultrasound scan to guide said tissue extraction.
61. A method of removing tissue pathologies utilizing a surgical device as described in clause 60 or any other clause wherein said scan guided tissue extraction is approximately real-time.
62. A method of removing tissue pathologies utilizing a surgical device as described in clause 51 or any other clause wherein said step of inserting a guide channel directed to the tissue pathology to be removed comprises the step of inserting a solid introducer rod having a diameter smaller that the lumen of said hollow cutting needle directed to the tissue pathology to be removed.
63. A method of removing tissue pathologies utilizing a surgical device as described in clause 62 or any other clause wherein said step of inserting a solid introducer rod having a diameter smaller that the lumen of said hollow cutting needle directed to the tissue pathology to be removed comprises the step of inserting said hallow cutting needle over said solid rod directed to the tissue pathology to be removed so as to allow said rod to pass through the lumen of said cutting needle.
64. A surgical device for tissue coring comprising:
   a hollow cutting needle having a beveled cutting edge along its distal boundary positioned within a rotatable support shaft internally situated within a handle element;
   at least one fastener element securing said cutting needle to with said rotatable support shaft;
   at least one rotatable turbine mechanically mated with said rotatable support shaft and responsive to a non-electromagnetic-field generating power source; and
   at least one core collection cylinder contiguous with said cutting needle and responsive an external negative pressure.
65. A surgical device for tissue coring as described in clause 64 or any other clause wherein said hollow cutting needle having a beveled cutting edge along its distal boundary comprises a hollow cutting needle having a serrated cutting edge along its distal boundary.
66. A surgical device for tissue coring as described in clause 64 or any other clause wherein said hollow cutting needle having a beveled cutting edge along its distal boundary comprises a hollow cutting needle having a blunt distal cap with split lateral apertures along its distal boundary to facilitate tissue side harvesting.
67. A surgical device for tissue coring as described in clause 64 or any other clause wherein said hollow cutting needle having a beveled cutting edge along its distal boundary comprises a hollow cutting needle having a beveled cutting edge along its distal boundary with a lumen diameter range of approximately 2 mm to 9 mm.
68. A surgical device for tissue coring as described in clause 64 or any other clause wherein said rotatable support shaft comprises a rotatable support shaft having a distally tapered end-section.
69. A surgical device for tissue coring as described in clause 68 or any other clause wherein said rotatable support shaft having a distally tapered end-section comprises a rotatable support shaft having a distally tapered end-section where said tapered end-section is circumferentially compressible.
70. A surgical device for tissue coring as described in clause 68 or any other clause wherein said rotatable support shaft having a distally tapered end-section comprises a rotatable support shaft having a distally tapered end-section where said tapered end-section is threaded so as to accept a corresponding fastener.
71. A surgical device for tissue coring as described in clause 64 or any other clause wherein said rotatable support shaft comprises a rotatable support shaft having a slotted penetration to accept a snap fastener.
72. A surgical device for tissue coring as described in clause 64 or any other clause wherein said rotatable support shaft comprises a rotatable support shaft having a slotted fastener leverage disengagement position.
73. A surgical device for tissue coring as described in clause 64 or any other clause wherein said fastener element securing said cutting needle to said rotatable support shaft comprises a compression fastener.
74. A surgical device for tissue coring as described in clause 64 or any other clause wherein said fastener element securing said cutting needle to said rotatable support shaft comprises a slotted snap fastener.
75. A surgical device for tissue coring as described in clause 64 or any other clause wherein said fastener element securing said cutting needle to said rotatable support shaft comprises a threaded fastener.
76. A surgical device for tissue coring as described in clause 75 or any other clause wherein said threaded fastener comprises a collet nut.
77. A surgical device for tissue coring as described in clause 64 or any other clause and further comprising a plurality of rotatable support shaft bearings.
78. A surgical device for tissue coring as described in clause 77 or any other clause wherein said plurality of rotatable support shaft bearings comprises a plurality of ceramic rotatable support shaft bearings.
79. A surgical device for tissue coring as described in clause 64 or any other clause wherein said handle element comprises at least one grip element coordinated with at least one end cap and at least one nose cap forming an internal turbine cavity.
80. A surgical device for tissue coring as described in clause 79 or any other clause wherein said nose cap comprises a nose cap selected from the group consisting of: a snap fitting nose cap, a lock fitting nose cap; and slide fitting nose cap.
81. A surgical device for tissue coring as described in clause 79 or any other clause wherein said grip element is secured to said end cap by a plurality of penetrating fasteners.
82. A surgical device for tissue coring as described in clause 79 or any other clause wherein said end cap comprises at least one detachable collection vessel slide lock position.
83. A surgical device for tissue coring as described in clause 64 or any other clause wherein said handle element comprises an integral external power source access position.
84. A surgical device for tissue coring as described in clause 83 or any other clause wherein said integral external power source access position comprises at least one air intake position.
85. A surgical device for tissue coring as described in clause 84 or any other clause wherein said air intake position comprises an air intake positioned substantially tangential to said rotatable turbine.
86. A surgical device for tissue coring as described in clause 64 or any other clause and further comprising at least one turbine muffler.
87. A surgical device for tissue coring as described in clause 86 or any other clause wherein said turbine muffler comprises at least one integral convoluted pathway.
88. A surgical device for tissue coring as described in clause 86 or any other clause wherein said turbine muffler comprises at least one nylon mesh insert.
89. A surgical device for tissue coring as described in clause 64 or any other clause wherein said handle element comprises a handle element having plurality of exhaust ports.
90. A surgical device for tissue coring as described in clause 64 or any other clause wherein said rotatable turbine comprises a rotatable turbine having plurality of turbine blade positions.
91. A surgical device for tissue coring as described in clause 64 or any other clause and further comprising at least one turbine bearing supporting said rotatable turbine.
92. A surgical device for tissue coring as described in clause 91 or any other clause wherein said turbine bearing comprises a ceramic turbine bearing.
93. A surgical device for tissue coring as described in clause 64 or any other clause wherein said rotatable turbine comprises a plastic rotatable turbine.
94. A surgical device for tissue coring as described in clause 64 or any other clause and further comprising at least one seal retainer plate.
95. A surgical device for tissue coring as described in clause 64 or any other clause and further comprising at least one cutting needle seal.
96. A surgical device for tissue coring as described in clause 95 or any other clause wherein said cutting needle seal comprises an o-ring and groove seal.
97. A surgical device for tissue coring as described in clause 64 or any other clause and further comprising at least one vacuum seal.
98. A surgical device for tissue coring as described in clause 64 or any other clause wherein said non-electromagnetic-field generating power source comprises a non-electromagnetic-field generating power source selected from the group consisting of: a pneumatic power source; and a hydraulic power source.
99. A surgical device for tissue coring as described in clause 64 or any other clause wherein said external negative pressure comprises a vacuum.
100. A surgical device for tissue coring as described in clause 99 or any other clause wherein said vacuum comprises an automatic motor generated vacuum and/or a manually generated vacuum.
101. A surgical device for tissue coring as described in clause 100 or any other clause wherein said manually generated vacuum comprises a manually generated vacuum through operation of a syringe having a retractable plunger element.
102. A surgical device for tissue coring as described in clause 64 or any other clause and further comprising at least one guide channel.
103. A surgical device for tissue coring as described in clause 102 or any other clause wherein said guide channel comprises a solid introducer rod having a diameter smaller than the lumen of said hollow cutting needle.
104. A surgical device for tissue coring as described in clause 64 or any other clause wherein said surgical device comprises a surgical device operable in the presence of an active X-ray scan, and/or active CT scan, and/or active MRI scan, and/or active ultrasound scan without causing interference in said scans.
105. A surgical device for tissue coring as described in clause 64 or any other clause wherein said core collection cylinder comprises a core collection cylinder having at least one cutting needle sealed insert aperture.
106. A surgical device for tissue coring as described in clause 64 or any other clause wherein said core collection cylinder comprises a core collection cylinder having at least one filter barrier.
107. A surgical device for tissue coring as described in clause 64 or any other clause wherein said core collection cylinder comprises a core collection cylinder having at least one fluid port.
108. A surgical device for tissue coring as described in clause 64 or any other clause wherein said core collection cylinder comprises a core collection cylinder having at least one external negative pressure port.
109. A surgical device for tissue coring as described in clause 108 or any other clause wherein said external negative pressure port comprises a sealed vacuum port.
110. A surgical device for tissue coring as described in clause 64 or any other clause wherein said core collection cylinder comprises a core collection cylinder having at least one separation membrane.
111. A surgical device for tissue coring as described in clause 64 or any other clause wherein said core collection cylinder comprises a core collection cylinder having at least one slide lock projection.
112. A surgical device for tissue coring as described in clause 64 or any other clause wherein said core collection cylinder comprises a core collection cylinder having at least one o-ring and groove seal securable to said hollow cutter needle.
113. A surgical device for tissue coring as described in clause 64 or any other clause wherein said core collection cylinder comprises a removable core collection cylinder.
114. A surgical device for tissue coring as described in clause 64 or any other clause wherein said core collection cylinder comprises a core collection cylinder responsive to a positive pressure.
115. A surgical device for tissue coring as described in clause 64 or any other clause wherein said core collection cylinder comprises a core collection cylinder penetrating a detachable collection vessel and sealed to the proximal end of cutting needle to accept an extracted tissue core.
116. A surgical device for tissue coring as described in clause 64 or any other clause wherein said core collection cylinder comprises a core collection cylinder and sealed to the proximal end of cutting needle to accept an extracted tissue core.
117. A surgical device for tissue coring as described in clause 64 or any other clause wherein said core collection cylinder comprises a core collection cylinder fitted to the end of said hollow cutting needle post-extraction to accept an extracted tissue core in response to an external positive pressure applied to the hollow cutting needle.
118. A surgical device for tissue coring as described in clause 64 or any other clause and further comprising a non-electromagnetic-field generating power source control element.
119. A surgical device for tissue coring as described in clause 64 or any other clause and further comprising an external negative pressure control element.
120. A method of tissue coring using a rotating needle device comprising the steps of:
   securing a hollow cutting needle having a beveled cutting edge along its distal boundary within a rotatable support shaft internally situated within a handle element;
   fastening said cutting needle to said rotatable support shaft;
   inserting a guide channel directed to the tissue to be cored;
   guiding said cutting needle through said guide channel to said tissue to be cored;
   actuating a rotatable turbine mechanically mated with said rotatable support shaft through application of a non-electromagnetic-field generating power source on said rotatable turbine;
   longitudinally penetrating said tissue with said cutting needle to a desired depth and accumulating a stratified cross-sectional sample of tissue within said cutting needle;
   deactivating said non-electromagnetic-field generating power source stopping rotation of said turbine;
   attaching a core collection cylinder contiguous with said cutting needle; and
   extracting said stratified cross-sectional sample of tissue from said cutting needle into said core collection cylinder by application of an external negative pressure.
121. A method of tissue coring using a rotating needle device as described in clause 120 or any other clause wherein said step of attaching a core collection cylinder contiguous with said cutting needle comprises the step of attaching a core collection cylinder contiguous with said cutting needle utilizing an o-ring and grove seal.
122. A method of tissue coring using a rotating needle device as described in clause 120 or any other clause wherein attaching a core collection cylinder contiguous with said cutting needle comprises the step of attaching a core collection cylinder contiguous with said cutting needle such that it penetrates a detachable collection vessel and applying an external negative pressure through said hollow cutting needle.
123. A method of tissue coring using a rotating needle device as described in clause 120 or any other clause wherein said step of attaching a core collection cylinder contiguous with said cutting needle comprises the step of attaching a core collection contiguous cylinder with said cutting needle such that it penetrates a therapeutic inducer secured to the proximal end of said cutting needle and applying an external negative pressure.
124. A method of tissue coring using a rotating needle device as described in clause 120 or any other clause wherein said step of extracting said stratified cross-sectional sample of tissue from said cutting needle comprises the step of attaching a core collection cylinder fitted to the proximal end of said cutting needle and applying an external negative pressure.
125. A method of tissue coring using a rotating needle device as described in clause 120 or any other clause wherein said step of extracting said stratified cross-sectional sample of tissue from said cutting needle comprises the step of removing said cutting needle and attaching said core collection cylinder to the distal end of said cutting needle and applying an external positive pressure.
126. A method of tissue coring using a rotating needle device as described in clause 125 or any other clause wherein said step of applying an external positive pressure through the proximal end of said cutting needle comprises the step of applying an automatic and/or manual external positive pressure through the proximal end of said cutting needle.
127. A method of tissue coring using a rotating needle device as described in clause 120 or any other clause wherein said step of guiding said cutting needle through said guide channel to said tissue to be cored comprises the step of utilizing the visual assistance of an active X-ray scan, and/or active CT scan, and/or active MRI scan, and/or active ultrasound scan to guide said cutting needle.
128. A method of tissue coring using a rotating needle device as described in clause 120 or any other clause wherein said step of longitudinally penetrating said tissue with said cutting needle to a desired depth and accumulating a stratified cross-sectional sample of tissue within said cutting needle comprises the step of utilizing the visual assistance of an active X-ray scan, and/or active CT scan, and/or active MRI scan, and/or active ultrasound scan to guide said cutting needle's penetration.
129. A method of tissue coring using a surgical device comprising the steps of:
   securing a hollow cutting needle having a beveled cutting edge along its distal boundary within a rotatable support shaft internally situated within a handle element;
   fastening said cutting needle to said rotatable support shaft;
   fastening a core collection cylinder responsive an external negative pressure to said cutting needle;
   inserting a guide channel directed to the tissue to be cored;
   activating said external negative pressure creating a vacuum within said cutting needle;
   actuating a rotatable turbine mechanically mated with said rotatable support shaft through application of a non-electromagnetic-field generating power source on said rotatable turbine;
   guiding said cutting needle through said guide channel;
   longitudinally penetrating said tissue with said cutting needle to a desired depth and removing a stratified cross-sectional sample of tissue within said cutting needle;
   accumulating said stratified cross-sectional sample of tissue within said core collection cylinder through action of said external negative pressure;
   de-activating said external negative pressure;
   deactivating said non-electromagnetic-field generating power source stopping rotation of said turbine; and
   extracting said stratified cross-sectional sample of tissue from said core collection cylinder.
130. A method of tissue coring using a surgical device as described in clause 129 or any other clause wherein said step of extracting said stratified cross-sectional sample of tissue from said core collection cylinder comprises the step of removing said core collection cylinder from said cutting needle and applying an external positive pressure into said core collection cylinder.
131. A method of tissue coring using a surgical device as described in clause 130 or any other clause wherein said step of applying an external positive pressure into said core collection cylinder comprises the step of applying a manual and/or automatic external positive pressure into said core collection cylinder.
132. A method of tissue coring using a surgical device as described in clause 129 or any other clause wherein said step of fastening a core collection cylinder responsive an external negative pressure to said cutting needle comprises the step of penetrating a collection vessel and/or a therapeutic inducer with a core collection cylinder to be affixed internally with the proximal end of said cutting needle.
133. A method of tissue coring using a surgical device as described in clause 129 or any other clause wherein said step of guiding said cutting needle through said guide channel comprises the step of utilizing the visual assistance of an active X-ray scan, and/or active CT scan, and/or active MRI scan, and/or active ultrasound scan to guide said cutting needle.
134. A method of tissue coring using a surgical device as described in clause 129 or any other clause wherein said step of longitudinally penetrating said tissue with said cutting needle to a desired depth and removing a stratified cross-sectional sample of tissue within said cutting needle comprises the step of utilizing the visual assistance of an active X-ray scan, and/or active CT scan, and/or active MRI scan, and/or active ultrasound scan to guide said penetration.
135. A system for delivering in situ therapeutic treatment using a rotating needle device comprising:
   a hollow cutting needle having a beveled cutting edge along its distal boundary positioned within a rotatable support shaft internally situated within a handle element;
   at least one fastener element securing said cutting needle with said rotatable support shaft;
   at least one rotatable turbine mechanically mated with said rotatable support shaft and responsive to a non-electromagnetic-field generating power source;
   at least one detachable therapeutic introducer having a delivery aperture fitted to the posterior aspect of said cutting needle; and
   at least one therapeutic treatment insert configured to pass into said delivery aperture and through said cutting needle so as to deposit said therapeutic treatment in situ.
136. A system for delivering in situ therapeutic treatment using a rotating needle device as described in clause 135 or any other clause wherein said detachable therapeutic introducer comprises a detachable therapeutic introducer having at least one slide lock projection.
137. A system for delivering in situ therapeutic treatment using a rotating needle device as described in clause 135 or any other clause wherein said detachable therapeutic introducer having a delivery aperture fitted to the posterior aspect of said cutting needle comprises a detachable therapeutic introducer having an o-ring and groove seal.
138. A system for delivering in situ therapeutic treatment using a rotating needle device as described in clause 135 or any other clause and further comprising a pressurized therapeutic treatment delivery apparatus.
139. A system for delivering in situ therapeutic treatment using a rotating needle device as described in clause 138 or any other clause wherein said pressurized therapeutic treatment delivery apparatus comprises a pressurized therapeutic treatment delivery apparatus selected from the group consisting of: an automatic pressurized therapeutic treatment delivery apparatus; and a manual pressurized therapeutic treatment delivery apparatus.
140. A system for delivering in situ therapeutic treatment using a rotating needle device as described in clause 135 or any other clause wherein said therapeutic treatment insert comprises a therapeutic insert selected from the group consisting of: irrigation fluid, anesthetic, cryo-probe; heat probe; laser probe; radioactive element; radioactive solution; chemotherapeutic agent; antigenic agent; pharmaceutical; cellular deposit; stem-cell deposit; stain; immunosuppressant; immuno-activator; anti-inflammatory; steroid; adjuvant; anti-biotic; growth factor; hormone; RF ablation element; biological marker; chemical marker; artificial material; buffer solution; and alcohol.
141. A system for delivering in situ therapeutic treatment using a rotating needle device as described in clause 135 or any other clause wherein said therapeutic treatment insert comprises at least one therapeutic treatment insert configured to be operable in the presence of an active X-ray scan, and/or active CT scan, and/or active MRI scan, and/or active ultrasound scan without causing interference in said scans.
142. A system for delivering in situ therapeutic treatment using a rotating needle device as described in clause 135 or any other clause and further comprising at least one core collection cylinder contiguous with said cutting needle and responsive an external negative pressure.
143. A system for delivering in situ therapeutic treatment using a rotating needle device as described in clause 135 or any other clause and further comprising at least one guide channel.
144. A system for delivering in situ therapeutic treatment using a rotating needle device as described in clause 143 or any other clause wherein said guide channel comprises a solid introducer rod having a diameter smaller that the lumen of said hollow cutting needle.
145. A method for delivering in situ therapeutic treatment using a rotating needle device comprising the steps of:
   securing a hollow cutting needle having a beveled cutting edge along its distal boundary within a rotatable support shaft internally situated within a handle element;
   fastening said cutting needle to said rotatable support shaft;
   attaching at least one therapeutic introducer having a delivery aperture fitted to the posterior aspect of said cutting needle;
   inserting at least one guide channel directed to the tissue to be treated;
   inserting said cutting needle through said guide channel;
   actuating a rotatable turbine mechanically mated with said rotatable support shaft through application of a non-electromagnetic-field generating power source on said rotatable turbine;
   penetrating said tissue with said cutting needle to a desired location; and
   inserting at least one therapeutic treatment through said delivery aperture and said cutting needle depositing said therapeutic treatment in situ.
146. A system for delivering in situ therapeutic treatment using a rotating needle device as described in clause 145 or any other clause wherein said step of inserting at least one therapeutic treatment through said delivery aperture and said cutting needle depositing said therapeutic treatment in situ comprises the step of inserting at least one therapeutic treatment selected from the group consisting of: irrigation fluid, anesthetic, cryo-probe; heat probe; laser probe; radioactive element; radioactive solution; chemotherapeutic agent; antigenic agent; stain; pharmaceutical; cellular deposit; stem-cell deposit; immunosuppressant; immuno-activator; anti-inflammatory; steroid; adjuvant; antibiotic; growth factor; hormone; RF ablation element; biological marker; chemical marker; artificial material; buffer solution; and alcohol.
147. A system for delivering in situ therapeutic treatment using a rotating needle device as described in clause 145 or any other clause wherein said step of inserting at least one therapeutic treatment through said delivery aperture and said cutting needle depositing said therapeutic treatment in situ comprises the step of injecting a therapeutic liquid.
148. A system for delivering in situ therapeutic treatment using a rotating needle device as described in clause 145 or any other clause wherein said step of inserting at least one therapeutic treatment through said delivery aperture and said cutting needle depositing said therapeutic treatment in situ comprises the step of inserting a probe.
149. A system for delivering in situ therapeutic treatment using a rotating needle device as described in clause 145 or any other clause wherein said step of inserting at least one therapeutic treatment through said delivery aperture and said cutting needle depositing said therapeutic treatment in situ comprises the step of inserting a therapeutic solid.
150. A system for delivering in situ therapeutic treatment using a rotating needle device as described in clause 145 or any other clause wherein said step of inserting at least one therapeutic treatment through said delivery aperture and said cutting needle depositing said therapeutic treatment in situ comprises the step of injecting a therapeutic gas.
151. A method for delivering in situ therapeutic treatment using a rotating needle device as described in clause 145 or any other clause wherein said step of inserting said cutting needle through said guide channel comprises the step utilizing the visual assistance of an active X-ray scan, and/or active CT scan, and/or active MRI scan, and/or active ultrasound scan to guide the insertion of said cutting needle through said guide channel.
152. A method for delivering in situ therapeutic treatment using a rotating needle device as described in clause 145 or any other clause wherein said step of penetrating said tissue with said cutting needle to a desired location comprises the step of utilizing the visual assistance of an active X-ray scan, and/or active CT scan, and/or active MRI scan, and/or active ultrasound scan to guide the penetration of said tissue with said cutting needle to a desired location.
153. A method for delivering in situ therapeutic treatment using a rotating needle device as described in clause 145 or any other clause wherein said step of inserting at least one therapeutic treatment comprises the step of utilizing the visual assistance of an active X-ray scan, and/or active CT scan, and/or active MRI scan, and/or active ultrasound scan to guide the insertion of at least one therapeutic treatment.
154. A method for delivering in situ therapeutic treatment using a rotating needle device as described in clause 145 or any other clause wherein said step of inserting at least one therapeutic treatment through said delivery aperture and said cutting needle depositing said therapeutic treatment in situ comprises the step of utilizing the visual assistance of an active X-ray scan, and/or active CT scan, and/or active MRI scan, and/or active ultrasound scan to guide deposition of said therapeutic treatment.
155. A method for delivering in situ therapeutic treatment using a rotating needle device as described in clause 145 or any other clause and further comprising the step of approximately real-time imaging said inserted therapeutic treatment utilizing an active X-ray scan, and/or active CT scan, and/or active MRI scan, and/or active ultrasound scan.
156. A method for delivering in situ therapeutic treatment using a rotating needle device as described in clause 145 or any other clause and further comprising the step of substantially removing said in situ therapeutic treatment.
157. A method for delivering in situ therapeutic treatment using a rotating needle device as described in clause 156 or any other clause wherein said step of removing said in situ therapeutic treatment comprises the step of generating an external negative pressure creating a vacuum force through said cutting needle substantially removing said in situ therapeutic treatment.
158. A method for delivering in situ therapeutic treatment using a rotating needle device as described in clause 145 or any other clause wherein said step inserting at least one guide channel directed to the tissue to be treated comprises the step of inserting a solid introducer rod having a diameter smaller that the lumen of said cutting needle directed to the tissue to be treated.
159. A method for delivering in situ therapeutic treatment using a rotating needle device as described in clause 158 or any other clause wherein said step of inserting a solid introducer rod having a diameter smaller that the lumen of said hollow cutting needle directed to the tissue to be treated comprises the step of inserting said hallow cutting needle over said solid rod directed to the tissue to be treated so as to allow said rod to pass through the lumen of said cutting needle.

While the invention has been described in connection with a preferred embodiment, it is not intended to limit the scope of the invention to the particular form set forth, but on the contrary, it is intended to cover such alternatives, modifications, and equivalents as may be included within the spirit and scope of the invention as defined by the statements of invention.

As can be easily understood from the foregoing, the basic concepts of the present invention may be embodied in a variety of ways. It involves both rotating needle surgical device apparatus embodiments as well as techniques of use as well as devices and techniques to accomplish the in situ delivery of therapeutic treatment. In this application, these embodiments are disclosed as part of the results shown to be achieved by the various devices described and as steps which are inherent to utilization. They are simply the natural result of utilizing the devices as intended and described. In addition, while some devices are disclosed, it should be understood that these not only accomplish certain methods but also can be varied in a number of ways. Importantly, as to all of the foregoing, all of these facets should be understood to be encompassed by this disclosure.

The discussion included in this application is intended to serve as a basic description. The reader should be aware that the specific discussion may not explicitly describe all embodiments possible; many alternatives are implicit. It also may not fully explain the generic nature of the invention and may not explicitly show how each feature or element can actually be representative of a broader function or of a great variety of alternative or equivalent elements. Again, these are implicitly included in this disclosure. Where the invention is described in device-oriented terminology, each element of the device implicitly performs a function. Apparatus claims may not only be included for the device described, but also method or process claims may be included to address the functions the invention and each element performs. Neither the description nor the terminology is intended to limit the scope of the claims that will be included in any subsequent patent application.

It should also be understood that a variety of changes may be made without departing from the essence of the invention. Such changes are also implicitly included in the description. They still fall within the scope of this invention. A broad disclosure encompassing both he explicit embodiment(s) shown, the great variety of implicit alternative embodiments, and the broad methods or processes and the like are encompassed by this disclosure and may be relied upon when drafting any claims. It should be understood that such language changes and broader or more detailed claiming may be accomplished at a later date (such as by any required deadline) or in the event the applicant subsequently seeks a patent filing based on this filing. With this understanding, the reader should be aware that this disclosure is to be understood to support any subsequently filed patent application that may seek examination of as broad a base of claims as deemed within the applicant's right and may be designed to yield a patent covering numerous aspects of the invention both independently and as an overall system.

Further, each of the various elements of the invention and claims may also be achieved in a variety of manners. Additionally, when used or implied, an element is to be understood as encompassing individual as well as plural structures that may or may not be physically connected. This disclosure should be understood to encompass each such variation, be it a variation of an embodiment of any apparatus embodiment, a method or process embodiment, or even merely a variation of any element of these. Particularly, it should be understood that as the disclosure relates to elements of the invention, the words for each element may be expressed by equivalent apparatus terms or method terms—even if only the function or result is the same.

Such equivalent, broader, or even more generic terms should be considered to be encompassed in the description of each element or action. Such terms can be substituted where desired to make explicit the implicitly broad coverage to which this invention is entitled. As but one example, it should be understood that all actions may be expressed as a means for taking that action or as an element which causes that action. Similarly, each physical element disclosed should be understood to encompass a disclosure of the action which that physical element facilitates. Regarding this last aspect, as but one example, the disclosure of a “fastener” should be understood to encompass disclosure of the act of “fastening”—whether explicitly discussed or not—and, conversely, were there effectively disclosure of the act of “fastening”, such a disclosure should be understood to encompass disclosure of a “element” and even a “means for fastening.” Such changes and alternative terms are to be understood to be explicitly included in the description.

Any patents, publications, or other references mentioned in this application for patent are hereby incorporated by reference. Any priority case(s) claimed by this application is hereby appended and hereby incorporated by reference. In addition, as to each term used it should be understood that unless its utilization in this application is inconsistent with a broadly supporting interpretation, common dictionary definitions should be understood as incorporated for each term and all definitions, alternative terms, and synonyms such as contained in the Random House Webster's Unabridged Dictionary, second edition are hereby incorporated by reference. Finally, all references listed in the list of References To Be Incorporated By Reference In Accordance With The Patent Application or other information statement filed with the application are hereby appended and hereby incorporated by reference, however, as to each of the above, to the extent that such information or statements incorporated by reference might be considered inconsistent with the patenting of this/these invention(s) such statements are expressly not to be considered as made by the applicant(s).

### REFERENCES TO BE INCORPORATED BY REFERENCE IN ACCORDANCE WITH THE PATENT APPLICATION

U.S. PATENTS

| | | Name of Patentee or Applicant |
|---|---|---|
| Pat. No. | Issue Date | of cited Document |
| 5,437,280 | 1995-08-01 | Hussman |
| 5,526,822 | 1996-06-18 | Burbank et al. |
| 5,564,436 | 1996-10-15 | Hakky et aI. |
| 5,916,229 | 1999-06-29 | Evans |
| 6,626,848B2 | 2003-09-30 | Neuenfeldt |
| 5,590,655 | 1977-01-07 | Hussaman |
| 5,769,086 | 1998-06-23 | Ritchart et al. |
| 4,874,376 | 1989-10-17 | Hawkins, Jr. |

U.S. PATENT APPLICATION PUBLICATIONS

| | | Name of Patentee or Applicant |
|---|---|---|
| Publication Number | Publication Date | of cited Document |
| 20060149163A1 | 2006-07-06 | Hibner et al. |
| 20070032742A1 | 2007-02-08 | Monson et al. |
| 20090112119A1 | 2009-04-30 | Kim |
| 20060229641A1 | 2006-10-12 | Gupta et al. |

FOREIGN PATENT DOCUMENTS

| | | | Name of Patentee |
|---|---|---|---|
| Foreign Document | | Publication | or Applicant |
| Number | Country Code | Date | of cited Document |
| 95/08293 | WO | 1995-03-30 | Hussman |

NON-PATENT LITERATURE DOCUMENTS

Burn, J. I. et al. Drill Biopsy and the Dissemination of Cancer,
Brit. J. Surg., 1968, Vol. 55, No. 8, August
Meyerowitz, B. R, et al. Drill Biopsy Confirmation of Breast Cancer,
Arch Surg-Vol 111. July 1976
Meyerowitz, B. R, et al. Pneumatic Drill for Tissue Biopsy,
American Journal of Surgery, Vol. 109, April 1965
US Provisional Application Number 61/147,359

Thus, the applicant(s) should be understood to have support to claim and make a statement of invention to at least: i) each of the rotating needle surgical devices and/or systems for delivery of therapeutic treatments as herein disclosed and described, ii) the related methods disclosed and described, iii) similar, equivalent, and even implicit variations of each of these devices and methods, iv) those alternative designs which accomplish each of the functions shown as are disclosed and described, v) those alternative designs and methods which accomplish each of the functions shown as are implicit to accomplish that which is disclosed and described, vi) each feature, component, and step shown as separate and independent inventions, vii) the applications enhanced by the various systems or components disclosed, viii) the resulting products produced by such systems or components, ix) each system, method, and element shown or described as now applied to any specific field or devices mentioned, x) methods and apparatuses substantially as described hereinbefore and with reference to any of the accompanying examples, xi) the various combinations and permutations of each of the elements disclosed, xii) each potentially dependent claim or concept as a dependency on each and every one of the independent claims or concepts presented, and xiii) all inventions described herein.

With regard to claims whether now or later presented for examination, it should be understood that for practical reasons and so as to avoid great expansion of the examination burden, the applicant may at any time present only initial claims or perhaps only initial claims with only initial dependencies. Other claims may be preserved within the specification as clauses. The office and any third persons interested in potential scope of this or subsequent applications should understand that broader claims may be presented at a later date in this case, in a case claiming the benefit of this case, or in any continuation in spite of any preliminary amendments, other amendments, claim language, or arguments presented, thus throughout the pendency of any case there is no intention to disclaim or surrender any potential subject matter. It should be understood that if or when broader claims are presented, such may require that any relevant prior art that may have been considered at any prior time may need to be re-visited since it is possible that to the extent any amendments, claim language, or arguments presented in this or any subsequent application are considered as made to avoid such prior art, such reasons may be eliminated by later presented claims or the like. Both the examiner and any person otherwise interested in existing or later potential coverage, or considering if there has at any time been any possibility of an indication of disclaimer or surrender of potential coverage, should be aware that no such surrender or disclaimer is ever intended or ever exists in this or any subsequent application. Limitations such as arose in Hakim v. Cannon Avent Group, PLC, 479 F.3d 1313 (Fed. Cir 2007), or the like are expressly not intended in this or any subsequent related matter. In addition, support should be understood to exist to the degree required under new matter laws—including but not limited to European Patent Convention Article 123(2) and United States Patent Law 35 USC 132 or other such laws—to permit the addition of any of the various dependencies or other elements presented under one independent claim or concept as dependencies or elements under any other independent claim or concept. In drafting any claims at any time whether in this application or in any subsequent application, it should also be understood that the applicant has intended to capture as full and broad a scope of coverage as legally available. To the extent that insubstantial substitutes are made, to the extent that the applicant did not in fact draft any claim so as to literally encompass any particular embodiment, and to the extent otherwise applicable, the applicant should not be understood to have in any way intended to or actually relinquished such coverage as the applicant simply may not have been able to anticipate all eventualities; one skilled in the art, should not be reasonably expected to have drafted a claim that would have literally encompassed such alternative embodiments.

Further, if or when used, the use of the transitional phrase “comprising” is used to maintain the “open-end” claims herein, according to traditional claim interpretation. Thus, unless the context requires otherwise, it should be understood that the term “comprise” or variations such as “comprises” or “comprising”, are intended to imply the inclusion of a stated element or step or group of elements or steps but not the exclusion of any other element or step or group of elements or steps. Such terms should be interpreted in their most expansive form so as to afford the applicant the broadest coverage legally permissible. The use of the phrase, “or any other claim” is used to provide support for any claim to be dependent on any other claim, such as another dependent claim, another independent claim, a previously listed claim, a subsequently listed claim, and the like. As one clarifying example, if a claim were dependent “on claim 20 or any other claim” or the like, it could be re-drafted as dependent on claim 1, claim 15, or even claim 715 (if such were to exist) if desired and still fall with the disclosure. It should be understood that this phrase also provides support for any combination of elements in the claims and even incorporates any desired proper antecedent basis for certain claim combinations such as with combinations of method, apparatus, process, and the like claims.

Furthermore, it should be noted that certain embodiments of the current invention may indicate a fastener, or the step of fastening. It should be noted that these may indicate a direct or in some cases an indirect connection and/or bringing together of disparate or non-disparate elements in a functional, non-functional or desired configuration.

In addition and as to computer aspects and each aspect amenable to software, programming or other electronic automation, the applicant(s) should be understood to have support to claim and make a statement of invention to at least: xvi) processes performed with the aid of or on a computer as described throughout the above discussion, xv) a programmable apparatus as described throughout the above discussion, xvi) a computer readable memory encoded with data to direct a computer comprising means or elements which function as described throughout the above discussion, xvii) a computer configured as herein disclosed and described, xviii) individual or combined subroutines and programs as herein disclosed and described, xix) the related methods disclosed and described, xx) similar, equivalent, and even implicit variations of each of these systems and methods, xxi) those alternative designs which accomplish each of the functions shown as are disclosed and described, xxii) those alternative designs and methods which accomplish each of the functions shown as are implicit to accomplish that which is disclosed and described, xxiii) each feature, component, and step shown as separate and independent inventions, and xxiv) the various combinations and permutations of each of the above.

Finally, any claims set forth at any time are hereby incorporated by reference as part of this description of the invention, and the applicant expressly reserves the right to use all of or a portion of such incorporated content of such claims as additional description to support any of or all of the claims or any element or component thereof, and the applicant further expressly reserves the right to move any portion of or all of the incorporated content of such claims or any element or component thereof from the description into the claims or vice-versa as necessary to define the matter for which protection is sought by this application or by any subsequent continuation, division, or continuation-in-part application thereof, or to obtain any benefit of, reduction in fees pursuant to, or to comply with the patent laws, rules, or regulations of any country or treaty, and such content incorporated by reference shall survive during the entire pendency of this application including any subsequent continuation, division, or continuation-in-part application thereof or any reissue or extension thereon.

1. A surgical device comprising:
a hollow cutting needle having a beveled cutting edge along its distal boundary positioned within a rotatable support shaft internally situated within a handle element;
at least one fastener element securing said cutting needle to said rotatable support shaft;
at least one rotatable turbine mechanically mated with said rotatable support shaft and responsive to a non-electromagnetic-field generating power source; and
at least one detachable collection vessel responsive an external negative pressure.

2-13. (canceled)

14. A surgical device as described in claim 1 and further comprising a plurality of rotatable support shaft bearings.

15. A surgical device as described in claim 14 wherein said plurality of rotatable support shaft bearings comprises a plurality of ceramic rotatable support shaft bearings.

16. A surgical device as described in claim 1 wherein said handle element comprises at least one grip element coordinated with at least one end cap and at least one nose cap forming an internal turbine cavity.

17-18. (canceled)

19. A surgical device as described in claim 16 wherein said end cap comprises at least one detachable collection vessel slide lock position.

20. A surgical device as described in claim 1 wherein said handle element comprises an integral external power source access position.

21-22. (canceled)

23. A surgical device as described in claim 1 and further comprising at least one turbine muffler.

24-25. (canceled)

26. A surgical device as described in claim 1 wherein said handle element comprises a handle element having a plurality of exhaust ports.

27. (canceled)

28. A surgical device as described in claim 1 and further comprising at least one turbine bearing supporting said rotatable turbine.

29-30. (canceled)

31. A surgical device as described in claim 1 and further comprising at least one seal retainer plate.

32. A surgical device as described in claim 1 and further comprising at least one cutting needle seal.

33. (canceled)

34. A surgical device as described in claim 1 and further comprising at least one vacuum seal.

35. A surgical device as described in claim 1 wherein said non-electromagnetic-field generating power source comprises a non-electromagnetic-field generating power source selected from the group consisting of: a pneumatic power source; and a hydraulic power source.

36. A surgical device as described in claim 1 wherein said detachable collection vessel comprises a detachable collection vessel having a sealed cutting needle insert aperture.

37. A surgical device as described in claim 1 wherein said detachable collection vessel comprises a detachable collection vessel having a fluid port.

38-42. (canceled)

43. A surgical device as described in claim 1 wherein said external negative pressure comprises a vacuum.

44. A surgical device as described in claim 43 wherein said vacuum comprises an automatic motor generated vacuum and/or a manually generated vacuum.

45-48. (canceled)

49. A surgical device as described in claim 1 and further comprising a non-electromagnetic-field generating power source control element.

50. A surgical device as described in claim 1 and further comprising an external negative pressure control element.

51-63. (canceled)

64. A surgical device for tissue coring comprising:
a hollow cutting needle having a beveled cutting edge along its distal boundary positioned within a rotatable support shaft internally situated within a handle element;
at least one fastener element securing said cutting needle to with said rotatable support shaft;
at least one rotatable turbine mechanically mated with said rotatable support shaft and responsive to a non-electromagnetic-field generating power source; and
at least one core collection cylinder contiguous with said cutting needle and responsive an external negative pressure.

65-80. (canceled)

81. A system for delivering in situ therapeutic treatment using a rotating needle device comprising:
a hollow cutting needle having a beveled cutting edge along its distal boundary positioned within a rotatable support shaft internally situated within a handle element;
at least one fastener element securing said cutting needle with said rotatable support shaft;
at least one rotatable turbine mechanically mated with said rotatable support shaft and responsive to a non-electromagnetic-field generating power source;
at least one detachable therapeutic introducer having a delivery aperture fitted to the posterior aspect of said cutting needle; and
at least one therapeutic treatment insert configured to pass into said delivery aperture and through said cutting needle so as to deposit said therapeutic treatment in situ.

82-91. (canceled)
